(19) United States
(12) Patent Application Publication
(10) Pub. No.: US 2013/0237746 A1
(43) Pub. Date: Sep. 12, 2013

(54) DEVICE FOR THE TREATMENT OF TINNITUS

(71) Applicant: Christian Veitl, Egling (DE)

(72) Inventor: Christian Veitl, Egling (DE)

(21) Appl. No.: 13/785,648

(22) Filed: Mar. 5, 2013

(30) Foreign Application Priority Data
Mar. 7, 2012 (DE) 102012101917.7

(51) Int. Cl. A61M 21/00

(52) U.S. Cl. CPC A61M 21/00
USPC 600/28

(57) ABSTRACT

The invention refers to a device for the treatment of tinnitus with an operating panel with a user interface and with at least one ear-plug connected to the operating panel for the input of a sound pattern consisting of sounds with sound levels in a frequency of more than 30 Hz into the ear's outer auditory canal. This device distinguishes itself particularly by the fact that the ear-plug is connected via a pressure tube to a pressure generator that generates pressure pulsations with a pulsation frequency of less than 10 Hz and a pressure difference of less than 1 bar, and that supplies the same through the pressure tube into the ear's outer auditory canal. Thus, it is possible to provide a device for the treatment of tinnitus with which better treatment results for a greater group of affected persons can be achieved.

The invention refers to a device for the treatment of tinnitus with an operating panel, a user interface, and at least one earplug connected to the user interface for the input of a sound pattern including sounds with sound levels in a frequency of more than 30 Hz into the ear's outer auditory canal.

The term “tinnitus” describes a phantom sound which is subjectively perceived by a person, although other persons in the vicinity cannot hear this sound. This tinnitus sound can be a serious strain for the persons concerned, and it can lead to concentration problems, insomnia, depression, and even panic. Various medical treatment approaches may focus upon the inner ear, the acoustic nerve or the acoustic center of the cerebral cortex, or the so-called auditory cortex. A given one of the various treatment methods may focus on one of these areas.

German patent DE 690 24 408 T2, for instance, shows an example that discloses a device for regulating the pressure in a person's acoustic meatus or outer auditory canal. This device comprises a body that is dimensioned and formed to be inserted into the person's outer auditory canal. It comprises an opening that enables the transport of fluids into and out of the acoustic meatus while the device is inserted, in order to generate a pressure herein. This device generates a selective static pressure comprising a lower absolute value than the ambient pressure. This pressure treatment effectuates a reduction or suppression of the tinnitus. However, a sustained elimination of the tinnitus could not be achieved. Furthermore this device was helpful only for a limited group of people.

The company ANM Adaptive Neuromodulation GmbH follows a different path with its acoustic CR®-Neuromodulation. This method of treatment considers the reason for the phantom sound to be pathologically synchronized, hyperactive articulated nerve cells in the brain. In a tightly focused way, the sterical arrangement of the tone frequency in the auditory cortex is utilized to accomplish the acoustic stimulation in the affected area. In alignment with the patient's tinnitus frequency determined in advance, appropriate impulses are calculated by means of a specific algorithm and then are programmed in chronological coordination with each other. Accordingly, the synchronous tinnitus activity in the auditory cortex can be disturbed. The stimulation occurs via medical ear-phones. By de-synchronizing the nerve cells, the pathological behavior of the nerve cells can be eliminated.

However, this treatment method is affected by the fact that the actual causes of tinnitus are not conclusively researched and, furthermore, can have different actuators. The stimulation of nerve cells by a sound pattern suggested here can be of help for patients only to a limited extent. In fact, in practice it shows that each condition of tinnitus is very subjective, has its own characteristic and is based on individual causes. Therefore, this treatment method lacks a holistic approach for the treatment of tinnitus thatis suitable for a larger number of affected people.

Therefore, the object of the invention is to create a device for the treatment of tinnitus that allows for better treatment results for a larger number of affected persons.

This object is achieved by a device with the features of claim 1. It specifically distinguishes itself by the fact that the ear-plug is connected via a pressure tube with a pressure generator that supplies pressure pulsations with a pulsation frequency of less than 10 Hz and with a pressure difference of less than 1 bar via the pressure tube into the outer auditory canal of the ear.

The invention is based upon the observation that a successful treatment of tinnitus requires a consideration of all interrelations as well as a complete identification of the affected areas in the body. Tinnitus generally affects four areas: the physical hearing organ or ear, the nerve pathways, the physical brain functions and the logical brain functions.

With tinnitus, the ear is obviously affected. The oldest analyses and research results regarding tinnitus are almost completely focused upon the ear. According to today's state of the art, there is every reason to believe that tinnitus in the ear can have various reasons or symptoms. Tinnitus can be caused by a trauma, a defect or an injury, for instance by too loud a noise or an impact, a shock wave, etc. It is also possible that inflammation can cause tinnitus, or that a circulatory disorder due to constriction or blockage, respectively acampsia or hardening of vessels is the cause of the sound in the ear.

According to the current state of research the affected areas in the ear are in the inner ear. In the cochlea, the hair cells or stereocilia can be affected or damaged by the causes mentioned. They form the interface towards the nerve cells or to the nerve pathway. Already upon consideration of the ear and the causes and the correlation with tinnitus it has to be established that often several symptoms and causes can apply, wherein it is very difficult to determine the exact correlation between cause and symptom.

Another affected area is the acoustic nerve that relays the hearing impulse of the hearing organ to the brain. Overstraining and/or inflammation of the acoustic nerve are considered one cause or one symptom of tinnitus.

Additionally, the physical brain function is affected. The relevant area in the brain is the auditory cortex, located laterally in the temporal lobe and processing the hearing impulse transmitted via the acoustic nerve. According to the current state of research, this area is largely affected by tinnitus. Possibly, even the cause of tinnitus may be located here.

Moreover, the logical brain function is of importance. Very early on it had been realized that tinnitus is not only caused by physical events, but that particularly mental aspects play an important role. Thus, stress is known to be a cause of tinnitus.

Within the framework of the invention it was realized that for a complete cure from tinnitus, the interaction of all these affected areas has to be considered. The complex interrelations are to be considered since, with chronic tinnitus, all four areas in the body are affected simultaneously. Furthermore, then as a rule what is the actual cause of tinnitus and what is now the symptom can no longer be established. From experience it has been realized that the symptoms can also take over the role of the cause, once they are developed. Then the symptom becomes the new cause, and the cause becomes the symptom.

This becomes clear with a concrete example. Stress can lead to overstraining the auditory cortex and the acoustic nerves, or to an inflammation and a circulatory disorder of the acoustic organ. On the other hand, a trauma in the acoustic organ can lead to overstraining the auditory cortex and the acoustic nerves and thus even to stress and depression. These interactions are manifold and are also verified by studies.

With the device according to the invention, for the first time all affected areas are treated simultaneously. The invention utilizes the following treatment components: air pressure massage of the acoustic organs, acoustic massage of the acoustic organs, stimulation of the acoustic nerves and of the acoustic cortex, and mental relaxation. In practical tests it turned out that, with the combinatory interaction of these treatment components, the healing results are particularly positive.

The healing effect of the individual treatment components is of particular importance. The air pressure massage of the acoustic organs represents a physical massage that mobilizes hardened and stiffened vessels. It increases the blood circulation and mobility and has a soothing effect on the patient.

The acoustic massage of the acoustic organs has a similar effect. However, it achieves the same in a different frequency range and with a finer structure. Thus, it also increases the blood circulation, has a soothing effect on the nerves and on the mental condition of the patient. It supplements the effect of the massage on the cochlea that is achieved by the air pressure massage, but it does not replace the same.

The feeling of pressure and the acoustic signals additionally stimulate the acoustic nerves and the acoustic cortex by aiming at the frequency ranges of the tinnitus noise.

Finally, the feeling of pressure by the air pressure massage increases, among others, the pouring out of endorphins and thus the mental relaxation of the patient. It also distracts him from the tinnitus noise. In particular, with stress and a patient's propensity to depression, this treatment component is of particular importance, as has been realized in the framework of the invention. Particularly, the acoustic signals have a relaxing effect.

At the same time, the device according to the invention combines all those treatment components in one single apparatus, so that a very simple and practical application of this treatment method can be achieved. In particular, the device according to the invention allows for a very stress-free and relaxed application.

Another advantage is that the device according to the invention can be taken along anytime and can be operated by any user.

Therefore, the device according to the invention enables a holistic treatment of tinnitus, thus being suited for virtually any patient and achieving reliably good therapy results.

Advantageous further embodiments of the device according to the invention are subject of the dependent claims.

For instance, the user interface may comprise a pressure controller or regulator to control the amount of pressure of the pressure pulsations. Then it is easily possible for the user to adjust the device according to the invention, so that he can achieve the best possible therapy result.

Further, the pressure difference of the pressure pulsations created by the pressure generator can amount to less than 0.5 bar. In practical tests it showed that here even better results can be achieved. The pressure difference can preferably be less than 0.2 bar and in particular lie between 0.05 and 0.1 bar, where the best therapy results have been achieved in the pre-tests.

If the pressure generator generates a pressure pulsation with excess pressure and low or negative pressure relative to the ambient pressure, the medicinal benefit is extremely good. In particular, a very good massage result and thus a stimulation of the blood circulation etc. can be achieved. Also, the acoustic nerves and the acoustic cortex can be particularly well stimulated.

It is a further advantage if the user interface comprises a frequency controller or regulator to control the pulsation frequency of the pressure pulsations. Then a user can adjust the pulsation frequency specifically to that range that gives him the best therapy result. Thus an individual adjustment to the respective patient is possible by himself or herself. In practice, however, the suitable pulsation frequency for the patient is initially determined and preset by a medical check-up. Thus a particularly tightly focused and individual treatment is achievable.

As pre-tests have shown, particularly good therapy results can be achieved when the pulsation frequency is less than 5 Hz. Preferably the pulsation frequency is between 0.25 and 5 Hz, wherein also here particularly good results have been achieved at a pulsation frequency of between 0.5 and 2 Hz.

It is a further advantage if the user interface comprises a control device or means for the adjustment of the volume of the sound and/or a specific sound pattern from a program matrix. Then a suitable and individual adjustment of the device for the respective patient is possible, wherein he can make additional individual adjustments in the course of the utilization.

Since the device according to the invention comprises a selection of ear-plugs of different sizes and/or shapes from which the user chooses the appropriate or adequate ear-plug, this is universally sellable merchandise. Each user can then make his or her individual adjustment without having to acquire additional elements. Therein an ear-plug is particularly well-suited for a user if it is adjusted to the form of the ear or the shape of the concha, resulting in best possible wear comfort simultaneously with optimized tightness.

It is further advantageous if the device according to the invention comprises a support means with which the at least one ear-plug is held in the user's ear. Thus the user's comfort is increased further. The support means is preferably adjustable to the size of the user's ear and/or head. This still further increases the user's comfort. In addition a particularly reliable function of the device according to the invention is achieved.

Here it is advantageous if the support means is a head holder encompassing the user's head. Then a particularly good wear comfort can be achieved. Furthermore, the at least one ear-plug is held in the user's ear particularly reliably. Preferably, two ear-plugs are applied so that, on both sides, an impact on the user's ear is attained. Thus particularly good therapy results can be achieved.

Hereafter the invention is explained in more detail by means of the figures of the drawings:

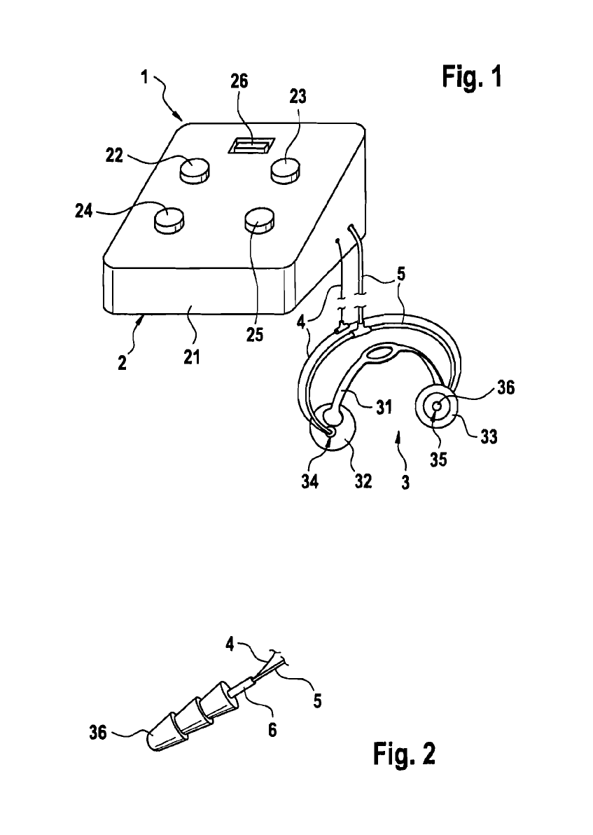

FIG. 1 shows a schematic view of the device according to the invention;

FIG. 2 shows a detailed view in the area of an ear-plug;

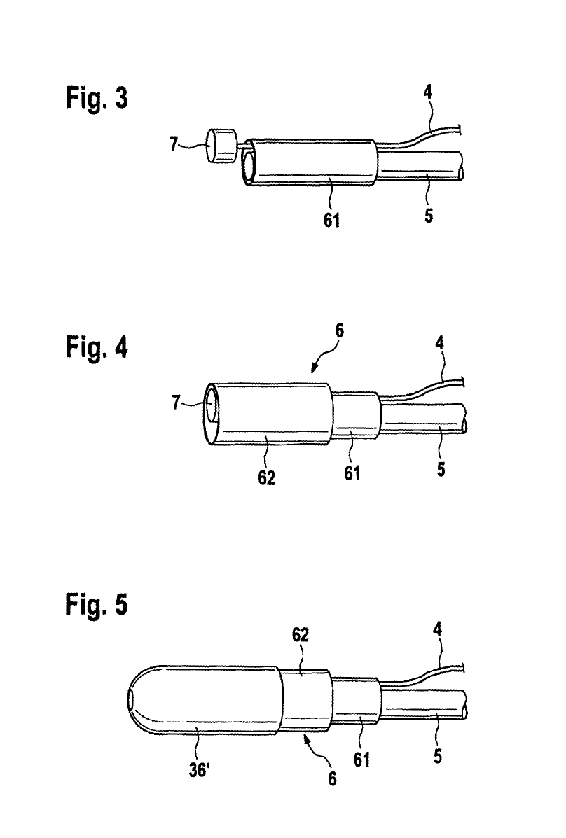

FIG. 3 shows a detailed view of the connection in a partly assembled state;

FIG. 4 shows the connection according to FIG. 3 in the totally assembled state; and

FIG. 5 shows the connection according to FIG. 4 with the ear-plug attached.

According to the depiction in FIG. 1, a device 1 for the treatment of tinnitus comprises an operating panel 2 as well as a head holder 3. These are connected via a speaker cable 4 and a flexible pressure tube 5.

The operating panel 2 comprises a housing 21 that is shown only schematically. On the top surface of the housing 21 a user surface or interface with various controllers for the individual setting of the desired parameters is arranged.

A pressure controller or regulator 22 is arranged on the user interface, by means of which the pressure of the pressure pulsation can be adjusted. In the example shown a pressure difference of the pressure pulsation can be continuously adjusted between 0 and 0.5 bar. This pressure regulator 22 interacts with a pressure generator (not shown) that provides a corresponding pneumatic pressure. Here the pressure generator is arranged in the operating panel 2.

Furthermore, a frequency controller or regulator 23 is positioned at the user interface, by means of which the pulsation frequency of the pressure pulsation is adjustable. In the shown embodiment the frequency regulator 23 allows for a pulsation frequency of up to 5 Hz.

Furthermore, a volume control 24 is arranged at the surface of the operating panel 2, by means of which the volume of the sounds and/or of a specific sound pattern from a program matrix is adjustable.

Furthermore, a program selection control 25 is provided, by means of which a user can freely select a specific sound or a specific sound pattern from a preset program matrix.

On the surface of the operating panel 2, furthermore, a data connection 26 is arranged which, in this case, is formed as a USB-connection. Hereto, for instance, an MP3 player can be connected if the pre-set sound pattern of the program matrix is not accessed.

The operating panel 2 is dimensioned in such a way that it can be carried without any problem. For this it is furnished with a clip (not shown) for fastening at a belt or the like. It is battery-operated and, additionally, a connection for direct access to a power supply system in the normal household area is provided. Thus, the device 1 can also be used stationary, respectively a recharge of the batteries or the accumulators in the operating panel 2 is possible.

The speaker cable 4 and the pressure tube 5 lead from the operating panel 2. They each split in the shown embodiment and are connected with both sides at the head holder 3. The latter comprises a mounting bracket or support means 31 as well as conchas 32 and 33. Thus, the head holder 3 is formed similarly to a conventional head holder for music systems or the like. However, the conchas 32 and 33 are formed in such a way that they comprise specific ear-plug receptions 34 and 35. Therein ear-plugs 36 can be inserted, of which only one is shown in FIG. 1. These ear-plugs 36 are locked in the conchas 32 and 33 such that they are positioned in a suitable manner in the acoustic meatus of the ear.

From FIG. 2 it is shown that such an ear-plug 36 is connected with the pressure tube 5 and the speaker cable 4 via a connection 6. The ear-plug 36 is formed in such a way that the air-pressure provided by the pressure tube 5 can act through the same into the acoustic meatus or outer auditory canal. The pressure tube 5 is designed to be flexible and generally does not expand with the applied pressure.

To apply the device 1 for treatment of tinnitus, a user selects suitable ear-plugs 36, fastens them at the ear-plug receptions 34 and 35, and, by the connection 6, connects them with the speaker cable 4 and the pressure tube 5. Then he attaches the head holder 3 on his head. Thereafter, he activates the device 1 by means of a switch (not shown) and selects the desired program from the program matrix with the program selection regulator 25. By means of the pressure regulator 22, the frequency control 23 and the volume control 24 he then adjusts these parameters in order to optimize the therapy result.

Thus the user receives an air pressure massage as well as an acoustic massage of the acoustic organs. Furthermore, the acoustic nerves and the acoustic cortex are stimulated, and a mental relaxation is achieved.

In FIGS. 3 to 5 the area of the connection 6 is shown in more detail. Therein FIG. 3 shows a partly assembled state of the connection 6. Here it can be seen that the speaker cable 4 leads to a speaker 7. The latter is of a particularly small size, in order to be suitable for the intended use. The pressure tube 5 ends with an open end next to the speaker cable 4 in the area of the speaker 7. Therein the speaker cable 4 and the pressure tube 5 are joined by a heat shrink tube 61.

In the depiction according to FIG. 4 the heat shrink tube 61 and the speaker 7 are furthermore covered by a sliding tube 62, so that the connection 6 is complete. Thus the speaker 7 and the open end of the pressure tube 5 are located in a suitable manner at the open end of the connection 6. Finally, according to the depiction in FIG. 5 a medical ear-plug 36′ is attached thereon, that is formed differently from the ear-plug 36 shown in FIG. 2. Now this arrangement can be inserted into one of the ear-plug receptions 34 and 35 respectively at the head holder 3.

The device 1 according to the invention for the treatment of tinnitus allows for further approaches at embodiments along with the embodiment explained above.

In a simplified embodiment it is therefore also possible to waive the pressure regulator 22. Then the device always works with the factory-provided pressure of the pressure pulsation.

Furthermore, in specific applications it can also be appropriate to provide the pressure difference of the pressure pulsation with a higher value than 0.5 bar. Here the individual requirements of the person to be treated have to be considered. Generally it can also be appropriate in specific applications to apply a pressure difference of less than 0.05 bar.

In a preferred embodiment of the invention, the pressure generator provides a pressure pulsation with excess pressure and low pressure relative to the ambient pressure. However, this is not necessary. The device 1 can also be operated exclusively in the low pressure range or the excess pressure range towards the ambient pressure.

Then the pressure pulsation is generated by build-up of pressure and discharge of pressure by the pressure generator.

In another simplified embodiment, the device 1 can also be used without the frequency control 23. Then the factory-provided pre-set pulsation frequency applies.

Also, with a view to the pulsation frequency it is possible with a view to the very different people affected by tinnitus, to apply a value of more than 5 Hz, if this should be appropriate in a particular case.

Further it is also possible to waive the volume control 24 in the framework of a simplified embodiment of the device 1. Then a factory-provided pre-set volume applies.

Also the program selection control 25 can be omitted in a simplified version of the device. Then a factory-provided pre-set sound or a certain pre-set sound pattern applies.

As is obvious from these explanations, individual or several or even all controls 22 to 25 can be omitted at the operating panel 2. However, in the embodiment according to FIG. 1 all controls 22 to 25 are installed.

Furthermore, it is also possible to apply a sound pattern from the most different external sources. This is not limited to the MP3 Player mentioned above, but can also use other data sources, like USB-sticks, internet connections, or the like, wherein the data connector 26 can also be equipped for different data connection systems than USB (like for instance SD etc.). As an alternative, it is, however, also possible to waive the data connector 26.

In an alternative embodiment, it is also possible to install two or more separate elements instead of one single operating panel 2. Thus, the pressure generator, with its control elements, can form a separate unit, as well as the sound source with its assigned control elements. Here it is only essential that in the user's ear region the pressure as well as the sound are available and take effect together.

In a preferred embodiment the device 1 comprises a selection of ear-plugs of different sizes and/or shapes, from which the user can choose the adequate ear-plug. However, this is not mandatory. Also a standard ear-plug can be applied here.

In a simplified embodiment of device 1 it is not essential to provide a head holder 3. Furthermore, one single ear-plug 36 can be sufficient, which is the case in particular if the tinnitus of the user appears only one-sided. Then this individual ear-plug 36 can be directly inserted into the ear without using a concha 32 or 33 or a mounting bracket 31.

On the other hand, with the application of the mounting bracket with the conchas 32 and 33, the physical form can be created such that these elements are customized to the head shape, as is known already from music systems. Furthermore, mounting brackets can be offered in various sizes.

Additionally it is also possible for an application of the device 1 on one side to equip the mounting bracket 31 with an ear-plug 36 only one-sided, so that the application is directed only to the one affected ear. On the other hand, it is also possible to treat the user's both ears not simultaneously, but alternately, which is advantageous for certain forms of tinnitus. Ultimately, the user's specific clinical picture is relevant for the specific application of device 1.

To support the treatment it is also possible to combine the application of the device 1 with the intake or infusion of medical substances or with stress-reducing measures.

1. A device for the treatment of tinnitus in a patient, wherein the device includes:
an operating panel with a user interface; and
at least one ear-plug connected to the operating panel and configured to input a sound pattern including sound levels with a frequency of more than 30 Hz into an outer auditory canal of the patient,
wherein the ear-plug is connected via a pressure tube with a pressure generator that generates pressure pulsations with a pulsation frequency of less than 10 Hz and a pressure difference of less than 1 bar and supplies the pressure pulsations through the pressure tube into the outer auditory canal of the patient.

2. The device of claim 1, wherein the user interface comprises a pressure regulator configured to control the pressure difference of the pressure pulsations.

3. The device of claim 1, wherein the pressure difference comprises less than 0.5 bar.

4. The device of claim 3, wherein the pressure difference comprises between 0.05 and 0.1 bar.

5. The device of claim 1, wherein the pressure generator generates a pressure pulsation with excess and low pressure relative to the ambient pressure.

6. The device of claim 1, wherein the user interface comprises a frequency regulator configured to control the pulsation frequency of the pressure pulsations.

7. The device of claim 1, wherein the pulsation frequency is less than 5 Hz.

8. The device of claim 7, wherein the pulsation frequency is between 0.25 and 5 Hz.

9. The device of claim 8, wherein the pulsation frequency is between 0.5 and 2 Hz.

10. The device of claim 1, wherein the user interface comprises a volume control configured to control a volume of the sound pattern.

11. The device of claim 1, wherein the sound pattern comprises a defined sound pattern from a program matrix.

12. The device of claim 11, wherein the user interface comprises a volume control configured to control a volume of the sound pattern.

13. The device of claim 1, further comprising a selection of ear plugs of different sizes.

14. The device of claim 1, further comprising a selection of ear plugs of different shapes.

15. The device of claim 1, further comprising a support configured to hold the at least one ear-plug is held in the ear of the patient.

16. The device of claim 15, wherein the support is adjustable to the size of the ear of the patient.

17. The device of claim 15, wherein the support is adjustable to the size of the head of the patient.

18. The device of claim 15, wherein the support comprises a head holder that encompasses the head of the patient.

19. The device of claim 1, further comprising a second ear-plug.
